(19) United States
(12) Patent Application Publication
(10) Pub. No.: US 2015/0164777 A1
(43) Pub. Date: Jun. 18, 2015

(54) Personal Care Products Containing Extracts of Rosemary

(71) Applicant: Kemin Industries, Inc., Des Moines, IA (US)

(72) Inventors: Shuting Wei, Ames, IA (US); Vandana Srivastava, Johnston, IA (US); Satish Nayak, West Des Moines, IA (US)

(73) Assignee: Kemin Industries, Inc., Des Moines, IA (US)

(21) Appl. No.: 14/104,722

(22) Filed: Dec. 12, 2013

(51) Int. Cl. A61K 8/97; A61Q 19/08

(52) U.S. Cl. CPC A61K 8/97; A61Q 19/08

(57) ABSTRACT

A method of reducing skin damage and/or providing health or cosmetic benefits in a subject, comprising application of a composition containing an effective amount of extracts of rosemary to the skin of the subject.

### BACKGROUND OF THE INVENTION

The present invention relates generally to personal care products containing extracts of rosemary and, more specifically, to the addition of extracts of rosemary to personal care products to protect the skin against damage due to exposure to ultraviolet radiation, provide skin soothing effects, improves skin conditioning and reduce the effects of skin aging.

Skin experiences constant exposure to environmental insult, including ultraviolet radiation (UV) exposure. The results of this exposure can be the accumulation of senescent keratinocytes. These keratinocytes can have a negative impact on the structure and function of the skin. Specifically, UV radiation on the skin can result in the formation of intracellular reactive oxygen species (ROS), which can cause DNA damage. This DNA damage acts as the stimulus to induce cellular senescence. The visual result of this process is aging. The first defense against this response is to utilize the antioxidants that are found in our bodies provided by healthy diet. Another opportunity to reduce the impact of this exposure is to utilize antioxidants applied topically on the skin via personal care products. Antioxidants have the ability to quench free radicals caused by the environment before they can cause a downstream effect.

Skin health can first be evaluated by physical appearance of the skin. Healthy skin appears hydrated, supple (soft, flexible) and smooth while poorly conditioned skin is typically marked by dry, scaly and rough appearance. Helping improve the look of poorly conditioned skin requires restoration of the epidermis to prevent loss of water. Restoration of the epidermis can be done using moisturizers that are topically applied to the skin. The use of antioxidants in moisturizers traditionally provides skin conditioning benefits.

Skin can undergo different types of environmental and physical stress on a daily basis, including exposure to irritating chemicals or UV radiation. As the body's natural response mechanism, epidermal keratinocytes can release a vast array of cytokines, such as interleukin 6 and 8 (IL-6 and IL-8). These cytokines can be used as the body's immune response to environmental exposure and are associated with the development of the irritation symptoms. These irritation symptoms can cause skin to become red and sore. Antioxidants help aid in soothing skin irritation.

### SUMMARY OF THE INVENTION

Extracts of rosemary have been discovered to have antioxidant effects that impact a wide spectrum of skin care issues. Included benefits are a decrease in the level of proteolytic enzymes, a decrease in inflammatory markers and a reduction in intracellular reactive oxygen species following exposure of the skin to UV radiation. Additional benefits include an increase in the synthesis of elastin and hyaluronic acid. The addition of these extracts of rosemary to personal care products provides a methodology for providing skin care benefits to users of the products, including a skin smoothing effect, a skin conditioning effect and anti-aging effects.

A purpose of the present invention is to provide personal care products containing extracts of rosemary to provide a wide range of beneficial effects to users of the products.

Another purpose of the present invention is to provide personal care products containing extracts of rosemary for use by persons at risk for skin damage.

Yet another purpose of the present invention is to provide a method of reducing skin damage in a subject, comprising application of a composition containing an effective amount of extracts of rosemary to the skin of the subject.

Still another purpose of the present invention is to provide a method of reducing the level of a proteolytic enzyme in a subject, comprising application of a composition containing an effective amount of extracts of rosemary to the skin of the subject.

A further purpose of the present invention is to provide a method of reducing the level of one or both of the protelytic enzymes MMP-1 and MMP-3.

Still a further purpose of the present invention is to provide a method of reducing the levels of an inflammatory marker in a subject, comprising application of a composition containing an effective amount of extracts of rosemary to the skin of the subject.

Yet a further purpose of the present invention is to provide a method of reducing the levels of one or both of the inflammatory markers IL-6 and IL-8.

Another purpose of the present invention is to provide a method of reducing the level of a reactive oxygen species in a subject, comprising application of a composition containing an effective amount of extracts of rosemary to the skin of the subject.

Still another purpose of the present invention is to provide a method of smoothing the skin of a subject, comprising application of a composition containing an effective amount of extracts of rosemary to the skin of the subject.

Yet another purpose of the present invention is to provide a method of soothing the skin of a subject, comprising application of a composition containing an effective amount of extracts of rosemary to the skin of the subject.

A further purpose of the present invention is to provide a method of increasing the level of synthesis of elastin in a subject, comprising application of a composition containing an effective amount of extracts of rosemary to the skin of the subject.

Still a further purpose of the present invention is to provide a method of increasing the level of synthesis of hyaluronic acid in a subject, comprising application of a composition containing an effective amount of extracts of rosemary to the skin of the subject.

These and other purposes of the present invention will be understood by those skilled in the art upon a review of this specification, the associated figures and the appended claims.

### BRIEF DESCRIPTION OF THE DRAWINGS

FIG. 1 is a chart of the results of the MTT assay of Example 1.

FIG. 2 is a chart of the results of the MMP-1 assay of Example 1.

FIG. 3 is a chart of the results of the MMP-3 assay of Example 1.

FIG. 4 is a chart of the results of the IL-6 assay of Example 1.

FIG. 5 is a chart of the results of the IL-8 assay of Example 1.

FIG. 6 is a chart of the results of the MTT assay of Example 2.

FIG. 7 is a chart of the results of the ELISA assay for elastin of Example 2.

FIG. 8 is a chart of the results of the ELISA assay for hyaluronic acid of Example 2.

FIG. 9 is a chart of the amount of reactive oxygen species produced for different treatments; each bar is an average of three different samples (n=3) and the error bars represent one standard deviation from the mean.

FIG. 10 is a series of photographs representing the reference patterns for different levels of scaliness.

### DETAILED DESCRIPTION OF PREFERRED EMBODIMENTS

Rosemary (Rosmarinus officinalis) is a perennial herb with fragrant needle-like leaves native to the Mediterranean region. Rosemary is one of many herbs in the family Labiatae. Certain cultivars of rosemary are rich in antioxidants, particularly carnosic acid and rosmarinic acid. A commercial source of antioxidants extracted from rosemary is Rosamox™ available from Kemin Industries, Inc. (Des Moines, Iowa) in a variety of forms and concentrations standardized to specified levels of carnosic acid.

### Example 1

### Effect of Rosamox™ on Fibroblast MMP-1, MMP-3, IL-6 and IL-8

### Purpose

This assay procedure is used to screen materials for their ability to reduce UVB induced increases in MMP-1, MMP-3, IL-6, IL-8 using cultured fibroblasts as the model.

### Summary of Test Method

Exposure of skin cells to UVB can result in an inflammatory response associated with the release of inflammatory markers such as IL-6, IL-8. In addition, UVB exposure can also result in the release of proteolytic enzymes such as MMP-1 and MMP-3. Both the release of inflammatory mediators and the proteolytic enzymes can have detrimental effects on the appearance and function of the skin. Therefore, material which can prevent these UVB induced responses can be beneficial as cosmetic ingredients.

In this study human dermal fibroblasts were grown in culture. The cells were treated for 24 hours after the UVB exposure At the end of the incubation period the cell culture media was collected and assayed for MMP-1, MMP-3, IL-6, IL-8 using ELISA based methods. Change in cell viability was also assessed using an MTT assay.

### Materials and Methods

Fibroblast Cell Culture

Human dermal fibroblasts were seeded into 24-well plates in Fibroblast Growth Medium (FGM) and grown at 37±2° C. and 5±1% CO₂ until confluent with a media change every 48 to 72 hours as needed. Once the cells were confluent they were treated with the test materials after the UVB exposure. During the 24 hours prior to the UVB exposure the cells were treated with DMEM media alone. For the UVB exposure the cell culture media was replaced with PBS and the cells were irradiated with 40 mJ/cm² UVB. After the UVB exposure the cells were treated with the test materials prepared in DMEM. At the end of the incubation period the cell culture media was collected and assayed for MMP-1, MMP-3, IL-6 and IL-8 while changes in cell viability was determined using an MTT assay.

MTT Assay

After the UVB incubation, the cell culture medium was removed (see above) and the fibroblasts were washed twice with PBS to remove any remaining test material. After the final wash, 500 μl of DMEM supplemented with 0.5 mg/ml MTT was added to each well and the cells were incubated for 1 hour at 37±2° C. and 5±1% CO₂. After the incubation, the DMEM/MTT solution was removed and the cells were washed again once with PBS and then 0.5 ml of isopropyl alcohol was added to the well to extract the purple formazin crystals. Two hundred microliters of the isopropyl extracts was transferred to a 96-well plate and the plate was read at 540 nm using isopropyl alcohol as a blank.

Preparation of ELISA Plates (MMP-1, MMP-3, IL-6, IL-8)

The ELISA plates were prepared by diluting the appropriate capture antibody in PBS. Next, 100 μl of the diluted capture antibody was added to the wells of a 96-well ELISA plate and the plate was incubated overnight at room temperature. On the following day the plate was washed three times with 300 μl wash buffer (0.05% Tween 20 in PBS) and then blocked by adding 300 μl of blocking buffer (1% BSA in PBS) to each well. The plate was incubated with the blocking buffer for at least one hour. After the incubation the blocking buffer was removed and the plate was washed three times as described above.

ELISA Procedure

A series of standards was prepared and 100 μl of each of these standards was dispensed into two wells (duplicates) in the appropriate 96-well plate. Subsequently, 100 μl of each sample was added to additional wells and the plate was incubated for two hours at room temperature. After the incubation the plate was washed three times as described above. Once the last wash was removed, 100 μl of a biotin conjugated detection antibody was added. After incubating the plate for two hours at room temperature the plate was washed again as described above. 100 μl of HRP-streptavidin was then added to each well and the plate was incubated for 20 minutes at room temperature. Once the last wash was removed, 100 μl of substrate solution (hydrogen peroxide+tetramethylbenzidine as a chromagen) was added to each well. Once a sufficient level of color development had occurred, 50 μl of stop solution (2N sulfuric acid) was added to each well and the plate was read at 460 nm.

Test Materials

The test materials consisted of Rosamox™, Lot#1307110701, at concentrations of 0.01%, 0.005%, and 0.001%.

### Results

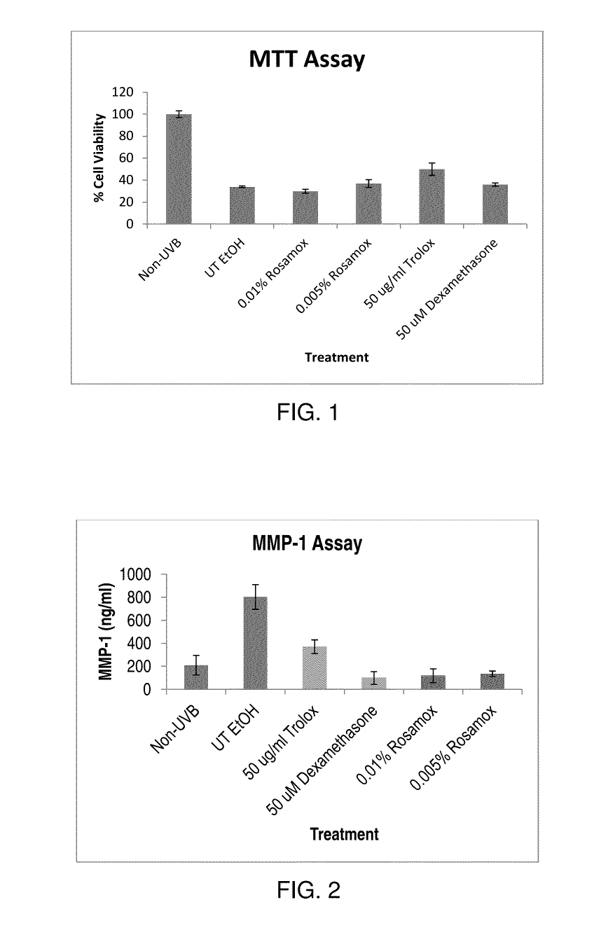

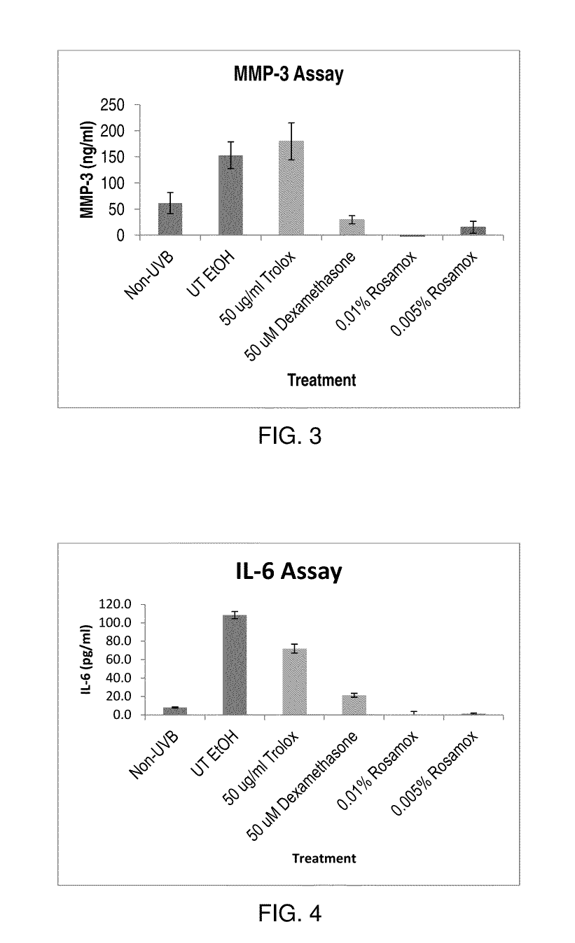

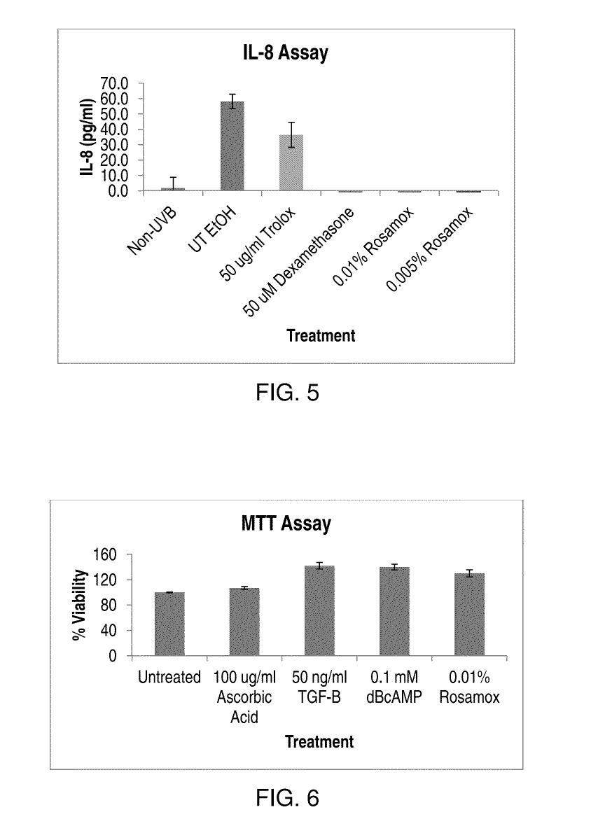

The results for the MTT assay are presented in FIG. 1. The values for this assay are expressed as mean viability±the standard deviation. The results for the MMP-1, MMP-3, IL-6, IL-8 assays are presented in Graphs 2-5, respectively. The values for these assays are presented as mean concentration±standard deviation.

### Discussion

UVB irradiation of the fibroblasts resulted in a significant decrease in the number of viable cells, along with a significant increase in all of the proteolytic and inflammatory markers measured in this study. The treatments with the test materials were not associated with any further decrease in the number of viable cells.

When Rosamox™ was added to the fibroblasts after the UVB exposure there was not a further decline in the number of viable cells and this material was observed to significantly decrease the release of MMP-1, MMP-3, IL-6 and IL-8.

### Example 2

### Effect of Rosamox™ on Elastin and Hyaluronic Acid

### Purpose

A fibroblast cell culture model was used to assess the ability of the test materials to exert an effect on elastin and hyaluronic acid synthesis. This study also assessed the viability of the cells after exposure to the test materials.

### Summary of Test Method

Fibroblasts are the main source of the extracellular matrix peptides, including the structural proteins collagen and elastin. Elastin is the main component of a network of elastic fibers that give tissues their ability to recoil after a transient stretch. This protein is released by fibroblasts (soluble elastin) into the extracellular space where it is then cross-linked to other elastin proteins to form an extensive network of fibers and sheets (insoluble elastin). Soluble elastin can be readily measured from cell culture medium via an ELISA based method.

Hyaluronan is a high molecular weight (1000-5000 kD) anionic polysaccharide. It is composed of repeating sets of disaccharides (glucuronate acetylglucosamine), which typically bind to a core protein and are synthesized extracellularly by a family of human hyaluronan synthase enzymes. Hyaluronan can be measured in cell or tissue culture media via a competitive ELISA based method.

Changes in cell number can be assessed via an MTT assay. The MTT assay is a colorimetric analysis of the metabolic activity of the cell, which is a reflection of the number of viable cells. Reduction of MTT by mitochondria results in the formation of insoluble purple formazin crystals that are extracted from the cells with isopropanol and quantified spectrophotometrically. The intensity of the purple color is directly proportional to the metabolic activity of the cells and inversely proportional to the toxicity of the test material

### Materials and Methods

Test Material Preparation

For use in cell culture, 1 gram of the materials was combined with either 10 ml of ultrapure water, 10 ml of ethanol or 10 ml of DMSO. The mixtures were then incubated for two hours at room temperature on a rocking platform. The obtained solution was then combined with the cell culture media for experimental use.

Preparation of Fibroblasts

Fibroblasts were seeded into the individual wells of a 24-well plate in 0.5 ml of Fibroblast Growth Media (FGM) and incubated overnight at 37±2° C. and 5±1% CO₂. On the following day the media was removed via aspiration to eliminate any non-adherent cells and replaced with 0.5 ml of fresh FGM. The cells were grown until confluent, with a media change every 48 to 72 hours. Upon reaching confluency the cells were treated for 24 hours with DMEM supplemented with 1.5% PBS to wash out any effects from the growth factors included in the normal culture media. After this 24-hour wash out period the cells were treated with the test materials at the specified concentrations dissolved in FGM with 1.5% PBS. Ascorbic acid (100 μg/ml) was used as a positive control for collagen synthesis, TGF-b (50 ng/ml) was used as a positive control for elastin, and dibutyrl cAMP (0.1 mM) was used as a positive control for hyaluronic acid. Untreated cells (negative controls) just received DMEM with 1.5% PBS. The cells were incubated for 48 hours and at the end of the incubation period cell culture medium was collected and either stored frozen (−75° C.) or assayed immediately. Materials were tested in triplicate.

MTT Assay

After the 2-day incubation, the cell culture medium was removed (see above) and the fibroblasts were washed twice with PBS to remove any remaining test material. After the final wash, 500 μl of DMEM supplemented with 0.5 mg/ml MTT was added to each well and the cells were incubated for 1 hour at 37±2° C. and 5±1% CO₂. After the incubation, the DMEM/MTT solution was removed and the cells were washed again once with PBS and then 0.5 ml of isopropyl alcohol was added to the well to extract the purple formazin crystals. Two hundred microliters of the isopropyl extracts was transferred to a 96-well plate and the plate was read at 540 nm using isopropyl alcohol as a blank.

Elastin ELISA Plate Preparation

Soluble α-elastin was dissolved in 0.1 M sodium carbonate (pH 9.0) at a concentration of 1.25 μg/ml. 150 μl of this solution was then applied to the wells of a 96-well maxisorp Nunc plate and the plate was incubated overnight at 4° C. On the following day the wells were saturated with PBS containing 0.25% BSA and 0.05% Tween 20. The plate was then incubated with this blocking solution for 1 hour at 37° C. and then washed two times with PBS containing 0.05% Tween 20.

Elastin Competitive ELISA

A set of α-elastin standards was generated ranging from 0 to 100 ng/ml. 180 μl of either standard or sample was then transferred to a 650 μl microcentrifuge tube. An anti-elastin antibody solution was prepared (the antibody was diluted 1:100 in PBS containing 0.25% BSA and 0.05% Tween 20) and 20 μl of the solution was added to the tube. The tubes were then incubated overnight at 4±2° C. On the following day, 150 μl was transferred from each tube to the 96-well elastin ELISA plate, and the plate was incubated for 1 hour at room temperature. The plate was then washed 3 times with PBS containing 0.05% Tween 20. After washing, 200 μl of a solution containing a peroxidase linked secondary antibody diluted in PBS containing 0.25% BSA and 0.05% Tween 20 was added, and the plate was incubated for 1 hour at room temperature. After washing the plate three times as described above, 200 μl of a substrate solution was added and the plate was incubated for 10 to 30 minutes in the dark at room temperature. After this final incubation the plate was read at 460 nm using a plate reader.

Hyaluronic Acid Assay

A series of hyaluronic acid standards was prepared ranging from 50 ng/ml to 3,200 ng/ml. Next, 100 μl of each standard (in duplicate) and sample was transferred to a well in an incubation plate. After adding 50 μl of detection solution to each well (except the reagent blank wells) the plate was incubated for 1±0.25 hour at 37±2° C. After the incubation, 100 μl of each sample/standard from the incubation plate was transferred to a corresponding well in the ELISA plate. The ELISA plate was covered and incubated for 30±5 minutes at 4° C. and then washed three times with 300 μl of wash buffer. After the final wash 100 μl of enzyme solution was added to each well and the plate was incubated at 37±2° C. for 30±5 minutes. After this incubation the wells were washed again as described above and then 100 μl of enzyme substrate solution was added to each well and the plate was incubated for 30-45 minutes at room temperature. After this final incubation 50 μl of stop solution was added to each well and the absorbance of the plate was measured at 405 nm using a plate reader.

### Calculations

MTT Assay

The mean MTT absorbance value for the negative control cells was calculated and used to represent 100% cell viability. The individual MTT values from the cells undergoing the various treatments were then divided by the mean value for the negative control cells and expressed as a percent to determine the change in cell viability caused by each treatment.

ELISA Assays

To quantify the amount of each substance present, a standard curve was generated using known concentrations of each substance. A regression analysis was performed to establish the line that best fits these data points. Absorbance values for the test materials and untreated samples were used to estimate the amount of each substance present in each sample.

Test Materials

The test materials consisted of Rosamox™, Lot#1307110701, at concentrations of 0.01%, 0.005%, and 0.001%.

### Results

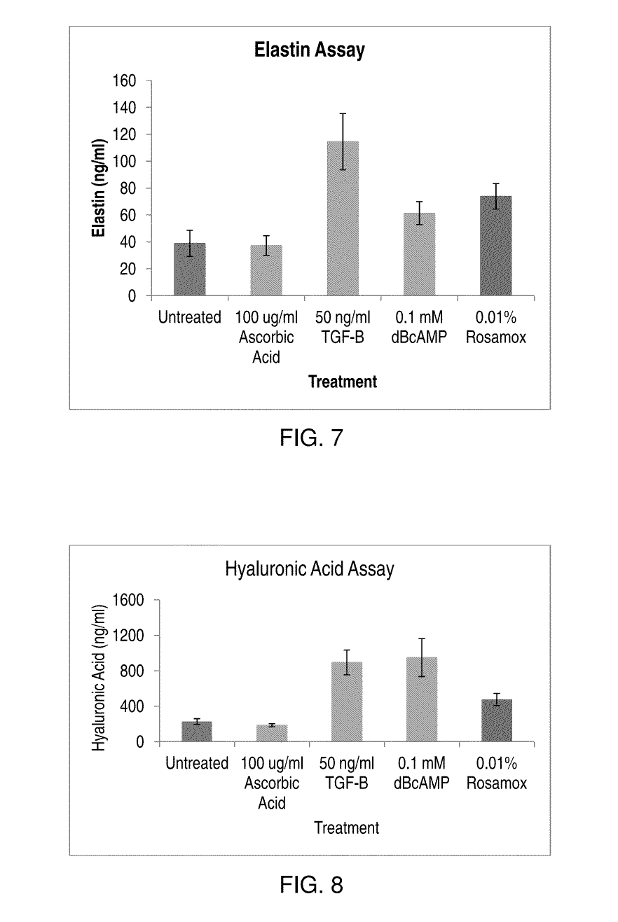

The results for the MTT assay are presented in FIG. 6. The values are presented as the mean percent viability±the standard deviation of the mean. The results for the ELISA assays are presented in FIG. 7 (Elastin), and FIG. 8 (Hyaluronic Acid). These values are also presented as mean concentration (ng/ml)±the standard deviation of the mean.

### Discussion

A fibroblast cell culture model was used to assess the ability of the test materials to exert an effect on elastin and hyaluronic acid synthesis. This study also assessed the viability of the cells after exposure to the test materials. In this study, treatment of the fibroblasts with the Rosamox™ test material was observed to increase the number of viable cells, increase elastin synthesis and also to increase hyaluronic acid synthesis.

### Example 3

### Elimination of Reactive Oxygen Species by Rosamox™

### Materials and Methods

In this study, human epidermal keratinocytes were seeded into culture flasks and grown at 37±2° C. and 5±1% CO₂ using Epilife® media for 24 hours and then incubated with 2′,7′-dichloro-dihydrofluorescein diacetate (DCF-CA) for 30 minutes at 37° C. DCF-DA is a commonly used dye and relatively non-fluorescent by nature. Once loaded into the cells, DCF will react with intracellular ROS to become highly fluorescent. The DCF-DA loaded cells were exposed to 1 J/cm² dose of UVB light. After the UVB exposure the keratinocytes were treated with the test materials and incubated for three hours at 37° C. and 5% CO₂. At the end of the treatment period media samples were obtained and assayed for the intracellular fluorescence with an excitation wavelength of 485 nm and an emission wavelength of 518 nm. Table 1 describes the various treatments used in this study.

TABLE 1
Treatments and related concentration used in the study

| Treatment | % Concentration |
|---|---|
| Untreated UVB Exposed | NA |
| Untreated Non-UVB Exposed | NA |
| Rosamox ™ | 0.01 |
| Rosamox ™ | 0.005 |
| Rosamox ™ | 0.001 |
| Vitamin E Acetate | 0.01 |
| Vitamin E Acetate | 0.005 |
| Vitamin E Acetate | 0.001 |
| 6-hydroxy-2,5,7,8-tetramethylchroman-2- | 0.0125 |
| carboxylic acid (Positive Control) | |

### Results

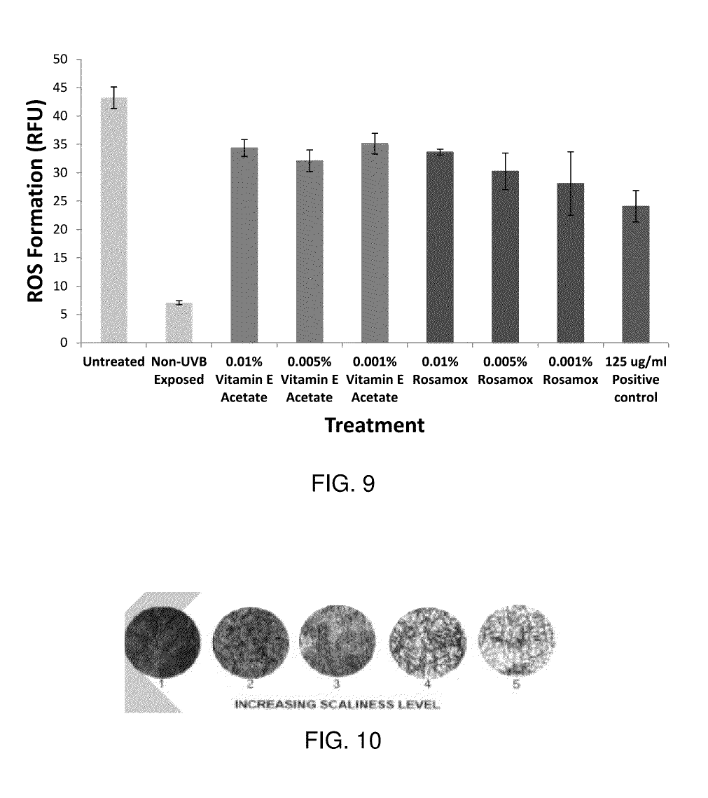

FIG. 9 illustrates the ability of the various treatments to reduce increases in intracellular ROS following exposure to UVB. The values are expressed as mean corrected RFU (relative fluorescence units)±standard deviation. It can be observed that the various Rosamox™ treatments reduced ROS formation. The Rosamox™ benefit was comparable to a commonly used skin antioxidant, Vitamin E acetate. As expected, the untreated sample experienced the highest amount of ROS formation as a result of UVB exposure.

Post treatment cells were evaluated, using MTT cytotoxicity assay. Results indicated no treatments induced any cell cytotoxicity.

### Discussion

Rosamox™ demonstrated the ability to quench reactive oxygen species (ROS) on the skin. The ability to quench free radicals may be the first opportunity to reduce the signs of aging. The observed results demonstrate that Rosamox™ can be used as an effective alternative to Vitamin E acetate to eliminate ROS on the skin and thus, may promote graceful aging. Rosamox™ can be added into formulation to provide a multi-functional benefit.

### Example 4

### Effect of Rosamox™ on Skin Conditioning

### Materials and Methods

Three skin cream formulations were made for this study. As shown in Table 2, Formula 1 had no active material and was used as a placebo control; while Formula 2 the active extract Rosamox™ was compared to Vitamin E acetate in Formula 3. This study was conducted on three subjects, each treated with one of the three formulations. The variation in conditioning of the skin was determined using D-Squame® discs applied to a clean, dry, skin surface. Discs were pressed firmly on the skin and then transferred to a black square on the storage card where they were compared with reference patterns. Skin condition is measured according to a published reference of scaliness patterns depicted in FIG. 10. Poorest condition skin with heavy amounts of scaling is assigned scaliness level 5, while normal skin producing small clumps of cells or a fine even single layer of cells is represented as level 1.

TABLE 2
Formulation of three test creams

| | | Formula 1 | Formula 2 | Formula 3 |
|---|---|---|---|---|
| Phase | Ingredients | % | % | % |
| A | Water | 72.2 | 71.7 | 71.7 |
| | Carbomer 940 | 0.5 | 0.5 | 0.5 |
| | Glycerin | 3.0 | 3.0 | 3.0 |
| B | Lysofix ™ Liquid - Glycerin | 3.5 | 3.5 | 3.5 |
| | (and) Glycine soja (Soybean) | | | |
| | Seed Extract | | | |
| C | Safflower oil | 12.0 | 12.0 | 12.0 |
| | Olive Oil | 6.0 | 6.0 | 6.0 |
| | Coco Caprylate | 2.0 | 2.0 | 2.0 |
| D | Sodium Hydroxide (33.33% | 0.2 | 0.2 | 0.2 |
| | Solution) to pH 5.5 | | | |
| E | Phenoxyethanol (and) | 0.6 | 0.6 | 0.6 |
| | Caprylyl Glycol | | | |
| F | Rosamox ™ - Helianthus | 0.0 | 0.5 | 0.0 |
| | annuus (Sunflower) Seed Oil | | | |
| | (and) Rosmarinus officinalis | | | |
| | (Rosemary) | | | |
| | Leaf Extract | | | |
| | Vitamin E Acetate | 0.0 | 0.0 | 0.5 |
| G | Water | up to | up to | up to |
| | | 100% | 100% | 100% |

### Results

Table 3 illustrates the change in level of scaliness, and hence conditioning effect, of the different treatments applied. Prior to treatment, skin of all test subjects was level 5. The short term (immediate) benefit of Rosamox™ for skin conditioning was observed as early as 1 hour post treatment, with skin recovering to level 1. The Rosamox™ benefit was similar to the commonly used skin conditioning agent Vitamin E acetate. An expected placebo effect was observed with the base formula which showed reduced scaliness from level 5 to level 3. Eight hours after application, the skin for all treatments was observed returning to its original scaliness level. The placebo control increased to level 4 while the other two treatments increased to level 3.

TABLE 3
Change in scaliness level as an effect of treatments applied

| | Scaliness Level by | | |
|---|---|---|---|
| | D-Squame ® Surface Sampling | | |
| | Baseline | 1 hour | 8 hours |
| | (Pre- | (Post- | (Post- |
| Formulation | Treatment) | Treatment) | Treatment) |
| Formula 1 (No active) | 5 | 3 | 4 |
| Formula 2 (Rosamox ™) | 5 | 1 | 3 |
| Formula 3 (Vitamin E acetate) | 5 | 1 | 3 |

### Discussion

Formulations with Rosamox™ exhibited complete improvement of skin condition to the smooth, youthful, non-scaly appearance of Level 1, better than the control, and equal to a recognized skin conditioner Vitamin E acetate. In addition, Rosamox™ continued to offer a sustained reduction of skin scaliness over eight hours post treatment. The observed results demonstrate that Rosamox™ may be used as an effective alternative to Vitamin E acetate for the relief of dry skin, helping skin improve to a smoother, more youthful appearance.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. A method of reducing skin damage in a subject, comprising application of a composition containing an effective amount of extracts of rosemary to the skin of the subject.

2. A method of reducing the level of a proteolytic enzyme in a subject, comprising application of a composition containing an effective amount of extracts of rosemary to the skin of the subject.

3. The method of claim 2, wherein the proteolytic enzyme is selected from the group consisting of MMP-1 and MMP-3.

4. A method of reducing the levels of an inflammatory marker in a subject, comprising application of a composition containing an effective amount of extracts of rosemary to the skin of the subject.

5. The method of claim 4, wherein the inflammatory marker is selected from the group consisting of IL-6 and IL-8.

6. A method of reducing the level of a reactive oxygen species in a subject, comprising application of a composition containing an effective amount of extracts of rosemary to the skin of the subject.

7. A method of smoothing the skin of a subject, comprising application of a composition containing an effective amount of extracts of rosemary to the skin of the subject.

8. A method of soothing the skin of a subject, comprising application of a composition containing an effective amount of extracts of rosemary to the skin of the subject.

9. A method of increasing the level of synthesis of elastin in a subject, comprising application of a composition containing an effective amount of extracts of rosemary to the skin of the subject.

10. A method of increasing the level of synthesis of hyaluronic acid in a subject, comprising application of a composition containing an effective amount of extracts of rosemary to the skin of the subject.
